(19) United States
(12) Patent Application Publication
(10) Pub. No.: US 2024/0268788 A1
(43) Pub. Date: Aug. 15, 2024

(54) ULTRASONIC PROBE, ULTRASONIC APPARATUS AND DETECTION METHOD

(71) Applicant: BOE Technology Group Co., Ltd., Beijing (CN)

(72) Inventors: Yonggang CAO, Beijing (CN); Xue DONG, Beijing (CN); Lei WANG, Beijing (CN); Yue GOU, Beijing (CN); Yuanyuan MA, Beijing (CN); Yue TONG, Beijing (CN); Yangbing LI, Beijing (CN); Yanling HAN, Beijing (CN); Wanzhi CHEN, Beijing (CN)

(21) Appl. No.: 18/646,703

(22) Filed: Apr. 25, 2024

Related U.S. Application Data
(63) Continuation of application No. PCT/CN2021/139272, filed on Dec. 17, 2021.

(51) Int. Cl. A61B 8/00; B06B 1/02; B06B 1/06; H10N 39/00

(52) U.S. Cl. CPC A61B 8/4488; A61B 8/54; B06B 1/0276; B06B 1/0611; B06B 1/064; H10N 39/00; B06B 2201/76

(57) ABSTRACT

Disclosed are an ultrasonic probe, an ultrasonic apparatus and a detection method. The ultrasonic probe includes: multiple transmitting transducers; multiple receiving transducers, each receiving transducer includes a receiving component and an ultrasonic control circuit electrically connected with the receiving component, and the multiple receiving components are distributed in an array; and multiple scanning signal lines and multiple readout signal lines, each of the scanning signal lines is located in a row gap between adjacent receiving components, each of the readout signal lines is located in a column gap between adjacent receiving components, multiple receiving components in the same row are electrically connected with the same scanning signal line by means of corresponding ultrasonic control circuits, and multiple receiving components in the same column are electrically connected with the same readout signal line by means of corresponding ultrasonic control circuits.

### CROSS-REFERENCE TO RELATED APPLICATIONS

The application is a continuation application of International Application No. PCT/CN2021/139272, filed Dec. 17, 2021, which is hereby incorporated by reference in its entirety.

### TECHNICAL FIELD

The disclosure relates to the technical field of the semiconductor, and in particular, relates to an ultrasonic probe, an ultrasonic apparatus and a detection method.

### BACKGROUND

A medical ultrasound imaging system mostly uses a linear array probe and a single-frequency scanning method to obtain an ultrasonic image of the target to be measured. The imaging resolution of this imaging system is often limited by the probe's operating frequency and the detection depth.

### SUMMARY

Embodiments of the present disclosure provide an ultrasonic probe, an ultrasonic apparatus, and a detection method. The ultrasonic probe, includes:
a plurality of transmitting transducers;
a plurality of receiving transducers, where each of the plurality of receiving transducers includes a receiving component and an ultrasonic control circuit electrically connected with the receiving component, and a plurality of receiving components are distributed in an array; and
a plurality of scanning signal lines and a plurality of readout signal lines, where the scanning signal line is located in a row gap between adjacent receiving components, the readout signal line is located in a column gap between adjacent receiving components, receiving components in a same row are electrically connected with a same scanning signal line by means of corresponding ultrasonic control circuits, and receiving components in a same column are electrically connected with a same readout signal line by means of the corresponding ultrasonic control circuits.

In some embodiments, each transmitting transducer includes: a first transmitting element which transmits a first acoustic wave signal, and at least one second transmitting elements which transmits a second acoustic wave signal; where a frequency of the first acoustic wave signal is less than a frequency of the second acoustic wave signal;
where the receiving component includes: a first receiving element which receives a third acoustic wave signal fed back according to the first acoustic wave signal, and a second receiving element which receives a fourth acoustic wave signal fed back according to the second acoustic wave signal.

In some embodiments, the first transmitting element and the second transmitting element are integrated in a same transmitting transducer; and the first receiving element and the second receiving element are integrated in a same receiving transducer.

In some embodiments, each transmitting transducer includes one first transmitting element, and a plurality of second transmitting elements; and the plurality of the second transmitting elements are distributed around the one first transmitting element.

In some embodiments, the receiving component includes: a first substrate; a first electrode on a side of the first substrate; a piezoelectric film layer on a side of the first electrode facing away from the first substrate; and a second electrode on a side of the piezoelectric film layer facing away from the first electrode.

In some embodiments, the ultrasonic control circuit is arranged between the first substrate and the first electrode; and the ultrasonic control circuit includes a first thin film transistor electrically connected with the receiving component, and the first electrode is electrically connected with a source of the first thin film transistor or a drain of the first thin film transistor.

In some embodiments, the first electrodes of different receiving components are independent of each other, and the second electrodes of the receiving components are an integrated structure.

In some embodiments, the plurality of transmitting transducers are distributed in an array; and a distribution density of the plurality of transmitting transducers is smaller than a distribution density of the plurality of receiving components.

In some embodiments, the first transmitting element and the at least one second transmitting element are independent of each other; and the first receiving element and the second receiving element are independent of each other;
where the first transmitting element and the first receiving element are an integrated structure, and the at least one second transmitting element and the second receiving element are an integrated structure.

In some embodiments, the receiving component includes: a second substrate; a third electrode on a side of the second substrate; a cavity on a side of the third electrode facing away from the second substrate; a diaphragm on a side of the cavity facing away from the third electrode; and a fourth electrode on a side of the diaphragm facing away from the cavity;
where a dimension of the cavity of the first receiving element in a direction parallel to the second substrate is greater than a dimension of the cavity of the second receiving element in the direction parallel to the second substrate.

In some embodiments, the ultrasonic control circuit is arranged between the second substrate and the third electrode; and
the ultrasonic control circuit includes a second thin film transistor electrically connected with the receiving component, and the third electrode is electrically connected with a source of the second thin film transistor or a drain of the second thin film transistor.

In some embodiments, the third electrodes of different receiving components are independent of each other; and the fourth electrodes of the receiving components are an integrated structure.

In some embodiments, a distribution density of the plurality of second receiving elements is greater than a distribution density of the plurality of first receiving elements.

An embodiment of the present disclosure also provides an ultrasonic apparatus, which includes the ultrasonic probe provided in the embodiments of the present disclosure, and further includes a processor; and the processor is electrically connected with the transmitting transducers and the receiving transducers, and configured to provide excitation signals to the transmitting transducers, and receive feedback signals fed back by the receiving transducers.

An embodiment of the present disclosure also provides a detection method for the ultrasonic probe provided in the embodiments of the present disclosure, which includes:
controlling the transmitting transducers to transmit ultrasonic signals;
loading scan signals line by line onto the scanning signal lines; and
obtaining feedback signals received by the receiving components and fed back according to the ultrasonic signals by means of the readout signal lines.

In some embodiments, operations of controlling the transmitting transducers to transmit ultrasonic signals; loading scan signals line by line onto the scanning signal lines; and obtaining feedback signals received by the receiving components and fed back according to the ultrasonic signals by means of the readout signal lines, include:
controlling a first transmitting element to transmit a first acoustic wave signal, loading a first scanning signal line by line to the scanning signal lines, and obtaining a third acoustic wave signal received by the first receiving element and fed back according to the first acoustic wave signal by means of the readout signal lines; where, the third acoustic wave signal includes location information of a target object; and
controlling a second transmitting element to transmit a second acoustic wave signal to the target object, loading a second scanning signal line by line to the scanning signal lines, and obtaining a fourth acoustic wave signal received by the second receiving element and fed back according to the second acoustic wave signal by means of the readout signal lines, to perform imaging according to information of the received fourth acoustic wave signal.

In some embodiments, the obtaining the fourth acoustic wave signal received by the second receiving element and fed back according to the second acoustic wave signal by means of the readout signal lines, includes:
collecting the fourth acoustic wave signal obtained by the second receiving element every first time interval, where the first time interval is less than half of a period of the fourth acoustic wave signal; and
obtaining a plurality of fourth acoustic wave signals obtained by each second receiving element at respective first time intervals by means of the readout signal lines, to determine related information of the target object.

In some embodiments, the determining the related information of the target object includes:
obtaining coordinates of the target object according to a following formula:

$${t}_{n} \times v = \sqrt{{( X - xt )}^{2} + {( Y - yt )}^{2} + {( Z - zt )}^{2}} + \sqrt{{( X - xn )}^{2} + {( Y - yn )}^{2} + {( Z - zn )}^{2}} ;$$

where, Tₓ(xₜ, yₜ, zₜ) is a center position of the transmitting transducer, tₙ is a sampling moment, and (xₙ, yₙ, zₙ) is coordinates of the receiving transducer that receives the signal at a moment tₙ.

### BRIEF DESCRIPTION OF FIGURES

FIG. 1 is a schematic diagram of the connection relationship between receiving transducers 2 and signal lines (scanning signal lines and readout signal lines).

FIG. 2 is a schematic diagram of top view distribution of transmitting transducers and receiving transducers provided by embodiments of the present disclosure.

FIG. 3 is a schematic cross-sectional view of FIG. 2 along a dotted line AB.

FIG. 4 is an enlarged schematic diagram of one of the transmitting transducers in FIG. 2.

FIG. 5 is another schematic diagram of the top view distribution of transmitting transducers and receiving transducers provided by embodiments of the present disclosure.

FIG. 6 is a schematic diagram of the cross-sectional distribution of transmitting transducers and receiving transducers provided by the embodiment of the present disclosure.

FIG. 7 is another schematic diagram of the cross-sectional distribution of transmitting transducers and receiving transducers provided by the embodiment of the present disclosure.

FIG. 8 is another schematic diagram of the top view distribution of transmitting transducers and receiving transducers provided by the embodiment of the present disclosure.

FIG. 9 is another schematic diagram of the top view distribution of transmitting transducers and receiving transducers provided by the embodiment of the present disclosure.

FIG. 10 is a schematic flow chart of an ultrasonic detection method provided by an embodiment of the present disclosure.

FIG. 11 is a schematic diagram of low-frequency ultrasonic rough scanning.

FIG. 12 is a low-frequency ultrasonic signal received by a receiving component at a certain moment (period).

FIG. 13 is a schematic diagram of high-frequency ultrasonic fine scanning.

FIG. 14 is a high-frequency ultrasonic signal received by a receiving component at a certain moment (period).

FIG. 15 is a schematic diagram of sampling signals many times of the receiving component array.

FIG. 16 is a schematic diagram of target positioning based on ultrasonic images.

### DETAILED DESCRIPTION

In order to make the purpose, technical solutions and advantages of embodiments of the present disclosure clearer, the technical solutions of embodiments of the present disclosure will be clearly and completely described below in conjunction with the drawings of embodiments of the present disclosure. Apparently, the described embodiments are some embodiments of the present disclosure, not all of them. Based on the described embodiments of the present disclosure, all other embodiments obtained by a person of ordinary skill in the art without creative efforts fall within the protection scope of the present disclosure.

Unless otherwise defined, the technical terms or scientific terms used in the present disclosure shall have the usual meanings understood by those skilled in the art to which the present disclosure belongs. “First”, “second” and similar words used in the present disclosure do not indicate any order, quantity or importance, but are only used to distinguish different components. Words such as “include” or “comprise” mean that the element or object appearing before the word includes the elements or objects listed after the word and their equivalents, without excluding other elements or objects. Words such as “connected” or “coupled” are not limited to physical or mechanical connections, but may include electrical connections, whether direct or indirect. “Up”, “down”, “left”, “right” and so on are only used to indicate the relative positional relationship; and when the absolute position of the described object changes, the relative positional relationship may also change accordingly.

To keep the following description of the embodiments of the present disclosure clear and concise, detailed descriptions of known functions and known components are omitted in the present disclosure.

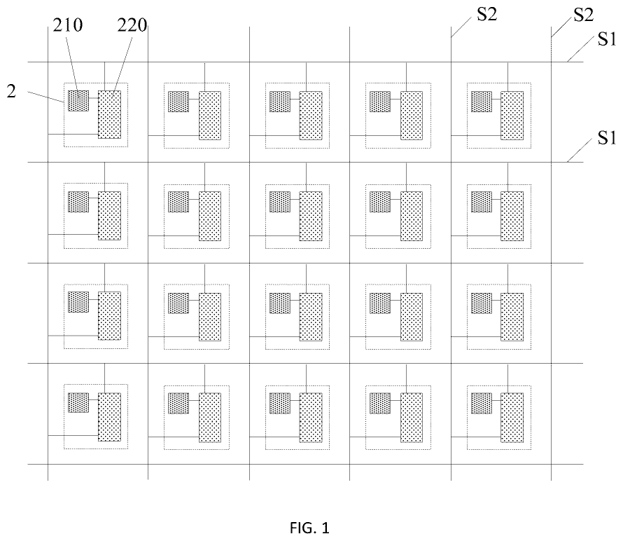

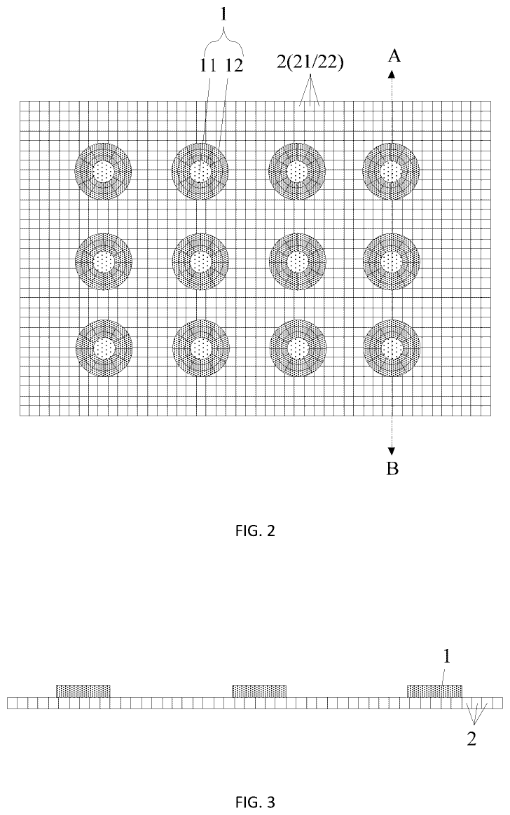

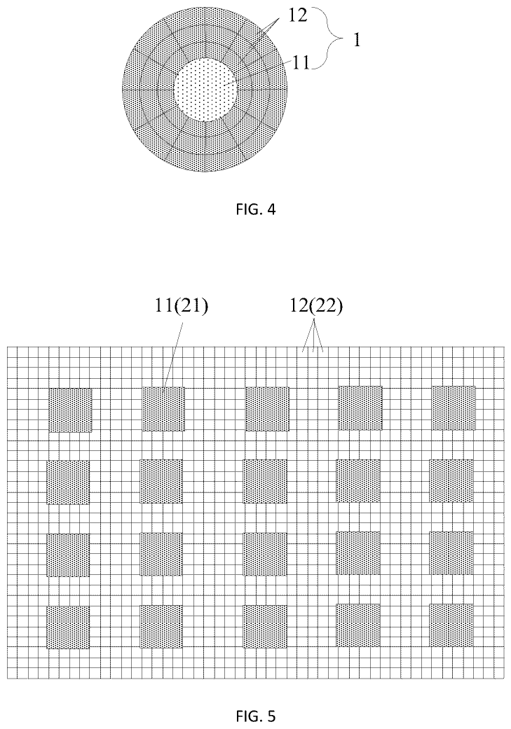

Embodiments of the present disclosure provide an ultrasonic probe, as shown in FIG. 1 to FIG. 5, where FIG. 1 is a schematic diagram of the connection relationship between receiving transducers 2 and signal lines (scanning signal lines and readout signal lines), FIG. 2 is a schematic diagram of distribution of transmitting transducers and receiving transducers, FIG. 3 is a schematic cross-sectional view of FIG. 2 along a dotted line AB, FIG. 4 is an enlarged schematic diagram of one of the transmitting transducers in FIG. 2, and FIG. 5 is another schematic diagram of distribution of transmitting transducers and receiving transducers. The ultrasonic probe includes:
a plurality of transmitting transducers 1;
a plurality of receiving transducers 2, where each receiving transducer 2 includes a receiving component 210 and an ultrasonic control circuit 220 electrically connected with the receiving component 210, and a plurality of receiving components 210 are distributed in an array; and
a plurality of scanning signal lines S1 and a plurality of readout signal lines S2, where the scanning signal line S1 is located in a row gap between adjacent receiving components 210, the readout signal line S2 is located in a column gap between adjacent receiving components 210, multiple receiving components 210 in the same row are electrically connected with the same scanning signal line S1 by means of the corresponding ultrasonic control circuits 220, and multiple receiving components 210 in the same column are electrically connected with the same readout signal line S2 by means of the corresponding ultrasonic control circuits 220.

In embodiments of the present disclosure, multiple receiving components 210 in the same row are electrically connected with the same scanning signal line S1 by means of the corresponding ultrasonic control circuits 220, and multiple receiving components 210 in the same column are electrically connected with the same readout signal line S2 by means of the corresponding ultrasonic control circuits 220. Compared with the traditional ultrasonic probe in which each receiving component needs to be connected with an independent signal line, in order to obtain the high image resolution, more signal lines are required, which eventually make the ultrasonic probe have a complex circuit and a large volume, the ultrasonic probe provided by the embodiment of the present disclosure can simplify the circuit of the ultrasonic probe while realizing ultrasonic images with the high image resolution, thereby simplifying the overall structure of the ultrasonic probe.

In some embodiments, as shown in FIG. 2, FIG. 4 and FIG. 5, each transmitting transducer 1 includes: a first transmitting element 11 which transmits a first acoustic wave signal, and at least one second transmitting elements 12 which transmits a second acoustic wave signal, where a frequency of the first acoustic wave signal is lower than a frequency of the second acoustic wave signal; and the receiving component 210 includes: a first receiving element 21 which receives the third acoustic wave signal fed back according to the first acoustic wave signal, and a second receiving element 22 which receives a fourth acoustic wave signal fed back according to the second acoustic wave signal. In some embodiments, the first acoustic wave signal may be a low-frequency acoustic wave signal, for example, the first acoustic wave signal may be an acoustic wave signal with a frequency in the range of 1 MHz to 2 MHz; and the second acoustic wave signal may be a high-frequency acoustic wave signal, for example, the second acoustic wave signal may be an acoustic wave signal with a frequency greater than 5 MHz. Correspondingly, the first transmitting element 11 can be a low-frequency transmitting element, and the second transmitting element 12 can be a high-frequency transmitting element; and the first receiving element 21 can be a low-frequency receiving element, and the second receiving element 22 can be a high-frequency receiving element.

In embodiments of the present disclosure, the transmitting transducer 1 includes a first transmitting element 11 for transmitting a first acoustic wave signal, and a second transmitting element 12 for transmitting a second acoustic wave signal; and the receiving component 210 includes a first receiving element 21, and a second receiving element 22. The ultrasonic probe is a high-low frequency composite structure. When performing ultrasonic detection, rough scanning of the size and position of the detection target is performed by means of the detection of the low-frequency ultrasonic wave; and based on the result of rough scanning; the high-frequency ultrasound is used for high-frequency high-resolution imaging in the local area, purposefully. Compared with the full-channel real-time sampling of the traditional ultrasonic probe, the ultrasonic probe provided by the embodiments of the present disclosure can reduce the working time and power consumption of the ultrasonic probe while achieving high-resolution imaging, to extend the use time of the ultrasonic probe.

In some embodiments, as shown in FIG. 2 or FIG. 4, the first transmitting element 11 and the second transmitting element 12 are integrated in the same transmitting transducer 1; and the first receiving element 21 and the second receiving element 22 are integrated in the same receiving transducer 2.

In some embodiments, as shown in FIG. 2 or FIG. 4, each transmitting transducer 1 includes one first transmitting element 11 and a plurality of second transmitting elements 12, and the plurality of second transmitting elements 12 are distributed around the first transmitting element 11. In some embodiments, the first transmitting element 11 can be located in the center, and adopts a whole piece of lead zirconate titanate piezoelectric ceramics (PZT) or perovskite type polycrystalline piezoelectric ceramics ((1-x)Pb(Mg_{1/3}Nb_{2/3})O₃₋ₓPbTiO₃, PMN-PT) piezoelectric material, which can be a circle, and the size of the circle diameter can be 2λ_{low} to 5λ_{low} (λ is the wavelength of the low-frequency ultrasound), to achieve the full range of emission/scanning. The plurality of second transmitting elements 12 are located outside the first transmitting element 11 and distributed in a ring-shaped structure. The plurality of second transmitting elements 12 can be in the shape of spherical shell focusing, and the plurality of second transmitting elements 12 can be in a phased array structure, which can realize the deflection of the focused beam by means of time delay control to realize high-frequency phased focusing. In some embodiments, the first transmitting element 11 and the second transmitting element 12 may be spaced independently of each other, and different second transmitting elements 12 may be spaced independently of each other.

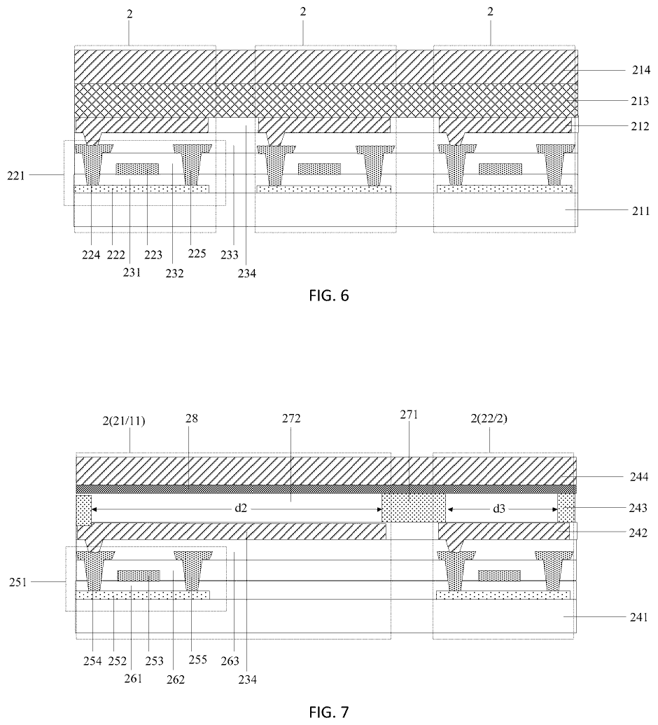

In some embodiments, as shown in FIG. 6, the receiving component 210 includes: a first substrate 211; a first electrode 212 on a side of the first substrate 211; a piezoelectric film layer 213 on a side of the first electrode 212 facing away from the first substrate 211; and a second electrode 214 on a side of the piezoelectric film layer 213 facing away from the first electrode 212. In some embodiments, the ultrasonic control circuit 220 may be arranged between the first substrate 211 and the first electrode 212; and the ultrasonic control circuit 220 includes a first thin film transistor 221 electrically connected with the receiving component 210, and the first electrode 212 is electrically connected with a source 224 or a drain 225 of the first thin film transistor 224. In this way, the integration of the receiving component 210 and the ultrasonic control circuit 220 is realized. In some embodiments, the material of the piezoelectric film layer 213 can be a piezoelectric polymer material, for example, it can be polyvinylidene fluoride (poly(1,1-difluoroethylene), PVDF) or polyvinylidene fluoride-trifluoroethylene copolymer (PVDF-TrFE). The receiving component 210 formed by the piezoelectric film layer 213 of this type of polymer material has broadband receiving performance, and then an ultrasonic control circuit is integrated to realize selective sampling of ultrasonic signals. In some embodiments, the distance d between the centers of two adjacent first electrodes 212 in the direction parallel to the first substrate 211 may be less than the half wavelength of the acoustic wave, where the sound velocity is selected to be 1540 m/s, which is the sound velocity of the acoustic lens material of the probe and human tissue. Since the medical ultrasonic frequency is several MHZ, when the frequency is 1MHZ, d is the largest, that is, specifically, d<sound velocity/frequency=1.5*10³/1*10⁶=1.5*10⁻³ m=1.5 mm, and d<half wavelength=0.75 mm. The distance d between the centers of two adjacent first electrodes 212 in the direction parallel to the first substrate 211 is relatively small, so that a refined two-dimensional receiving array structure is realized to have a higher imaging image resolution.

In some embodiments, the ultrasonic control circuit 220 may also include other thin film transistors and capacitors, where the first transistor 221 is a thin film transistor in the ultrasonic control circuit 220 electrically connected with the receiving component 210; and the ultrasonic probe may also include other signal lines, which is not limited in the disclosure. In some embodiments, the circuit of the ultrasonic control circuit 220 may be the same or similar to the structure of the pixel circuit in the display panel, or may also be the same or similar to the circuit structure of the fingerprint recognition device.

In some embodiments, as shown in FIG. 6, the first thin film transistor 221 may include: a first active layer 222 located on a side of the first substrate 211; a first insulating layer 231 located on a side, away from the first substrate 211, of the first active layer 222; a first gate 223 located on a side, away from the first active layer 222, of the first insulating layer 231; a second insulating layer 232 located on a side, away from the first insulating layer 231, of the first gate 223; and a first source 224 and a first drain 225 located on a side, away from the first gate 223, of the second insulating layer 232. A third insulating layer 234 may also be located between the first source 224 and the first electrode 212.

In some embodiments, as shown in FIG. 6, the first electrodes 212 of different receiving components 210 are independent of each other, and the second electrodes 214 of various receiving components 210 are an integrated structure.

In some embodiments, as shown in FIG. 2, the plurality of transmitting transducers 1 are distributed in an array; and the distribution density of the plurality of transmitting transducers 1 is smaller than the distribution density of the plurality of receiving components 210.

In some embodiments, as shown in FIG. 5, the first transmitting element 11 and the second transmitting element 12 are independent of each other; the first receiving element 21 and the second receiving element 22 are independent of each other; the first transmitting element 11 and the second receiving element 21 are an integrated structure; and the second transmitting element 12 and the second receiving element 22 are an integrated structure. In this way, the transceiver transducer is realized as an integrally manufactured device.

In some embodiments, the first transmitting element 11 (the first receiving element 21) can be used as a high-frequency transceiver transducer unit, and the second transmitting element 12 (the second receiving element 22) can be used as a low-frequency transceiver transducer unit. The high-frequency transceiver transducer unit and the low-frequency transceiver transducer unit can be the same in the corresponding film thickness, but can be different in the diameter and side length, etc., to realize the differentiation of the operating frequency of the device.

In some embodiments, when the first transmitting element 11 and the first receiving element 21 are an integrated structure, and the second transmitting element 12 and the second receiving element 22 are an integrated structure, as shown in FIG. 7, the receiving component 210 includes: a second substrate 241; a third electrode 242 on a side of the second substrate 241; a cavity 272 on a side of the third electrode 242 facing away from the second substrate 241; a diaphragm 28 on a side of the cavity 272 facing away from the third electrode 242; and a fourth electrode 244 on a side of the diaphragm 28 facing away from the cavity; where the dimension d2 of the cavity 272 of the first receiving element 21 (the first transmitting element 11) in the direction parallel to the second substrate 241 is greater than the dimension d3 of the cavity 272 of the second receiving element 22 (the second transmitting element 12) in the direction parallel to the second substrate 241.

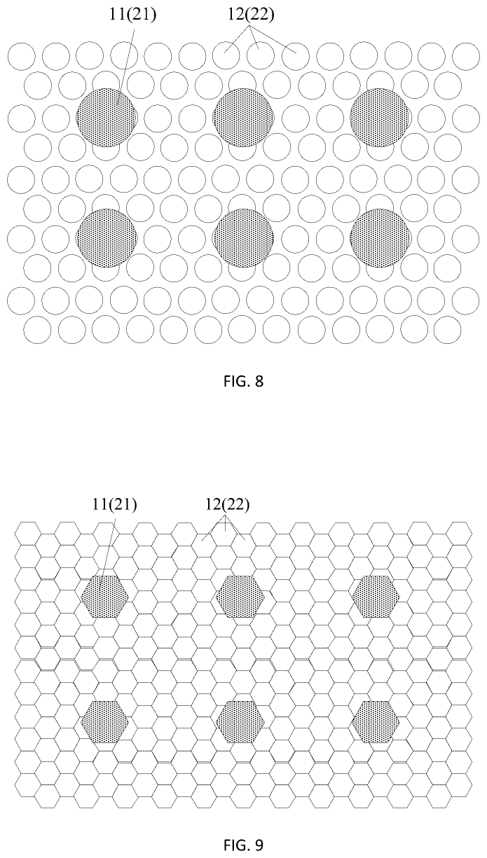

In some embodiments, a shape of the orthographic projection of the cavity 272 on the second substrate 241 can be a circle as shown in FIG. 8, can also be a square as shown in FIG. 5, and can also be a regular hexagon as shown in FIG. 9. Among the cavities 272 of the same shape, the cavity 272 with a smaller size belongs to a high-frequency transducer, and the cavity 272 with a larger size belongs to a low-frequency transducer.

In some embodiments, as shown in FIG. 7, the ultrasonic control circuit 220 is arranged between the second substrate 241 and the third electrode 242; and the ultrasonic control circuit 220 includes a second thin film transistor 251 electrically connected with the receiving component, and the third electrode 242 is electrically connected with the source 254 or the drain 255 of the second thin film transistor 251. In this way, the integration of the receiving component 210 and the ultrasonic control circuit 220 is realized.

In some embodiments, in the ultrasonic probe structure shown in FIG. 7, the ultrasonic control circuit 220 may also include other thin film transistors and capacitors, where the second thin film transistor 251 is a thin film transistor in the ultrasonic control circuit 220 electrically connected with the receiving component 210; and the ultrasonic probe may also include other signal lines, which is not limited in the disclosure. In some embodiments, the circuit of the ultrasonic control circuit 220 may be the same or similar to the structure of the pixel circuit in the display panel, or may also be the same or similar to the circuit structure of the fingerprint recognition device.

In some embodiments, as shown in FIG. 6, the second thin film transistor 251 may include a second active layer 252 located on a side of the second substrate 241; a fourth insulating layer 261 located on a side, away from the second substrate 241, of the second active layer 252; a second gate 253 located on a side, away from the second active layer 252, of the fourth insulating layer 261; and a fifth insulating layer 262 located on a side, away from the fourth insulating layer 261, of the second gate 253; and a second source 254 and a second drain 255 located on a side, away from the second gate 253, of the fifth insulating layer 262. A sixth insulating layer 263 may also be located between the second source 254 and the third electrode 242.

In some embodiments, as shown in FIG. 7, the third electrodes 242 of different receiving components 210 are independent of each other; and the fourth electrodes 244 of various receiving components 210 are an integrated structure.

In some embodiments, as shown in FIG. 5, FIG. 8 or FIG. 9, the distribution density of the plurality of second receiving elements 22 is greater than the distribution density of the plurality of first receiving elements 21.

Based on the same inventive concept, embodiments of the present disclosure further provide an ultrasonic apparatus, which includes the ultrasonic probe provided by the embodiments of the present disclosure, and also includes a processor; and the processor is electrically connected with the transmitting transducer and the receiving transducer, and configured to provide the excitation signal to the transmitting transducer and receive the feedback signal fed back by the receiving transducer.

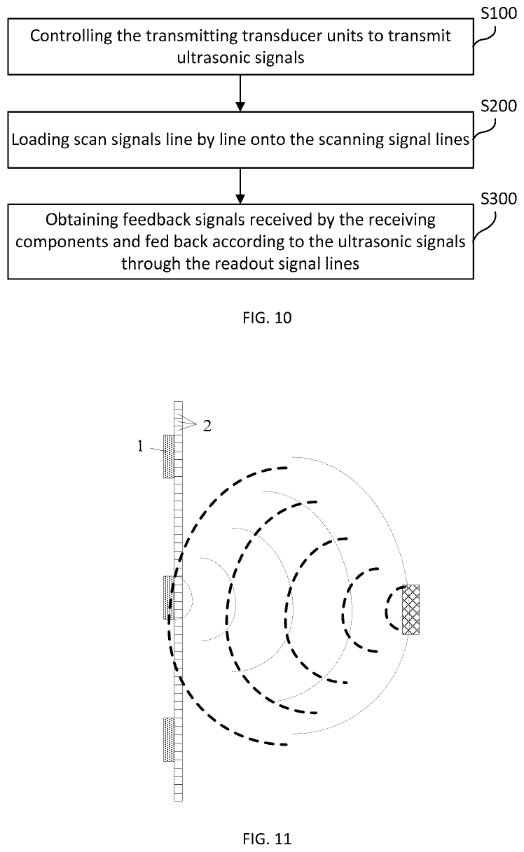

Based on the same inventive concept, as shown in FIG. 10, embodiments of the present disclosure further provide a detection method for an ultrasonic probe provided by embodiments of the present disclosure, which includes:
step S100, controlling the transmitting transducers to transmit ultrasonic signals;
step S200, loading scan signals line by line onto the scanning signal lines; and
step S300, obtaining feedback signals received by the receiving components and fed back according to the ultrasonic signals by means of the readout signal lines.

In some embodiments, the detection method provided by embodiments of the present disclosure: operations of controlling the transmitting transducers to transmit ultrasonic signals; loading scan signals line by line onto the scanning signal lines; and obtaining feedback signals received by the receiving components and fed back according to the ultrasonic signals by means of the readout signal lines, may include:
controlling the first transmitting element to transmit the first acoustic wave signal, loading the first scanning signal line by line to the scanning signal lines, and obtaining the third acoustic wave signal received by the first receiving element and fed back according to the first acoustic wave signal by means of the readout signal lines; where, the third acoustic wave signal includes location information of a target object; and
controlling the second transmitting element to transmit the second acoustic wave signal to the target object, loading the second scanning signal line by line to the scanning signal lines, and obtaining the fourth acoustic wave signal received by the second receiving element and fed back according to the second acoustic wave signal by means of the readout signal lines, to perform imaging according to information of the received fourth acoustic wave signal.

In some embodiments, obtaining the fourth acoustic wave signal received by the second receiving element and fed back according to the second acoustic wave signal by means of the readout signal lines, may include:
collecting the fourth acoustic wave signal obtained by the second receiving element every first time interval, where the first time interval is less than half of a period of the fourth acoustic wave signal; and obtaining a plurality of fourth acoustic wave signals obtained by each second
receiving element at respective first time intervals by means of the readout signal lines, to determine related information of the target object.

In embodiments of the present disclosure, compared with the full-channel real-time sampling of the traditional two-dimensional ultrasonic imaging system, the embodiment of the present disclosure will perform discontinuous “slicing” sampling on the reflected ultrasonic echo based on the ultrasonic control circuit integrated in the two-dimensional array, and finally realize the “ultrasonic imaging” of the detection target by obtaining the “ultrasonic image” of the signal.

In some embodiments, determining the related information of the target object may include: obtaining coordinates of the target object according to the following formula:

$${t}_{n} \times v = \sqrt{{( X - xt )}^{2} + {( Y - yt )}^{2} + {( Z - zt )}^{2}} + \sqrt{{( X - xn )}^{2} + {( Y - yn )}^{2} + {( Z - zn )}^{2}} ;$$

where, Tₓ(xₜ, yₜ, zₜ) is a center position of the transmitting transducer, tₙ is a sampling moment, and (xₙ, yₙ, zₙ) is the coordinates of the receiving transducer that receives the signal at a moment tₙ.

In order to more clearly understand the detection method provided by embodiments of the present disclosure, the following specific description is given.

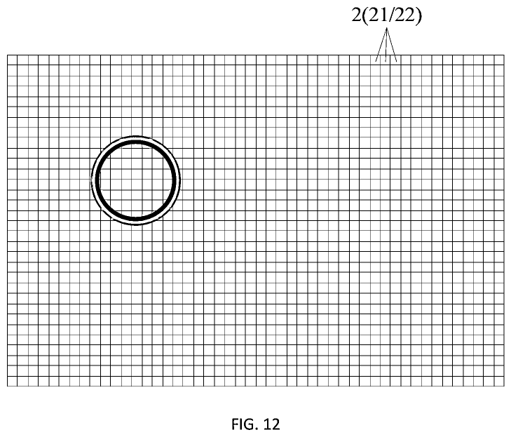

Rough scanning: the low-frequency ultrasonic transducer emits wide-beam scanning acoustic waves to quickly scan the area to be tested, to obtain the size and location information of the target object, as shown in FIG. 11 below. In this case, the receiving component receives the signal at a certain moment (period) as shown in FIG. 12.

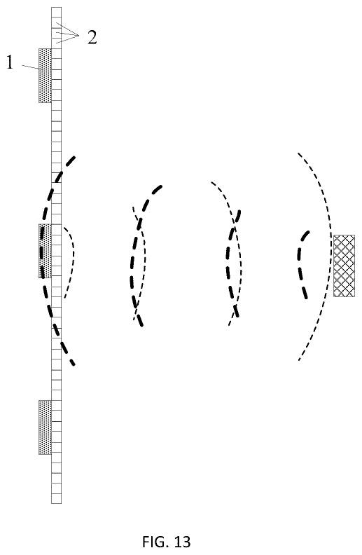

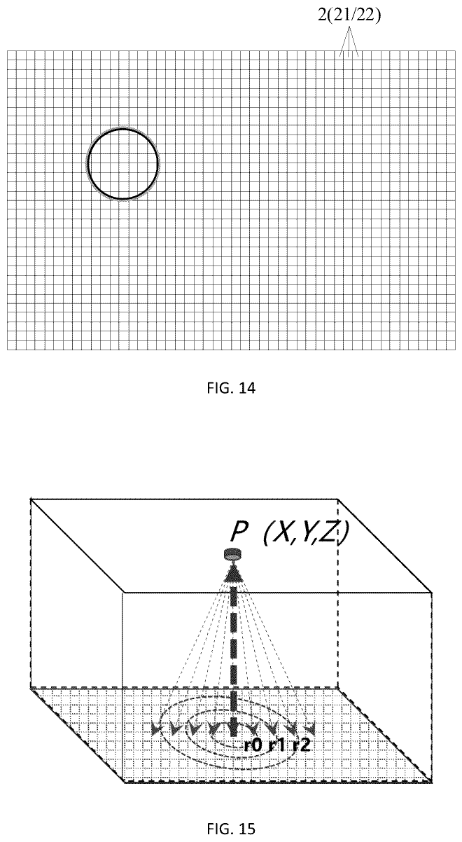

Fine scanning: the high-frequency ultrasonic transducer emits a focused beam at a fixed point based on the size and location information of the target object obtained by the rough scanning, and the beam width ranges from 2 mm to 3 mm, as shown in FIG. 13 below. In this case, the receiving component receives the signal at a certain moment (period) as shown in FIG. 14.

Echo signal collection: the ultrasonic control circuit integrated in the device is used to select any time point t1\t2\t3 . . . within the time tₛₜₐᵣₜ˜t_{end}, and perform integral collection of the signal, herein the integral time is less than T/2 (T is the period of the ultrasonic signal, and the reciprocal of the frequency); and finally, according to the size of the signal received by each receiving component, the ultrasonic image can be obtained, which reflects the spatial distribution characteristics of the ultrasonic wave, but the restoration scale of the wave front is affected by the size of the array element.

In some embodiments, regarding the sampling method, when the target object P(X, Y, Z) is detected, the corresponding ultrasonic images are obtained at time t1\t2\t3, as shown in FIG. 15, herein, r1\r2\r3 respectively represent the radii of the obtained ultrasonic images.

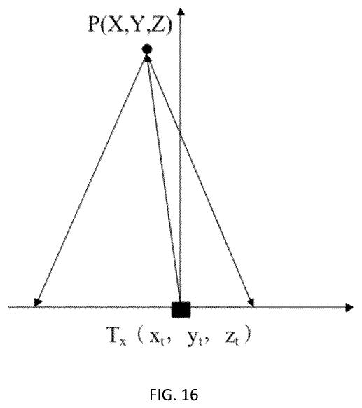

According to the geometric relationship shown in FIG. 16, the solution to the coordinates (X, Y, Z) of point P can be realized, and the solution formula is as follows:

$${t}_{n} \times v = \sqrt{{( X - xt )}^{2} + {( Y - yt )}^{2} + {( Z - zt )}^{2}} + \sqrt{{( X - xn )}^{2} + {( Y - yn )}^{2} + {( Z - zn )}^{2}} ;$$

where, Tₓ(xₜ, yₜ, zₜ) is a center position of the transmitting transducer, tₙ is a sampling moment, and (xₙ, yₙ, zₙ) is the coordinates of the receiving transducer that receives the signal at a moment tₙ. For larger targets, the imaging of the target object can be achieved by mechanical/phased scanning of reflected ultrasonic beams.

While preferred embodiments of the disclosure have been described, those skilled in the art may make additional changes and modifications to these embodiments once the basic inventive concepts are known. Therefore, the appended claims are intended to be construed to include the preferred embodiments as well as all changes and modifications that fall within the scope of the present disclosure.

Apparently, those skilled in the art can make various modifications and variations to the embodiments of the present disclosure without departing from the spirit and scope of the embodiments of the present disclosure. Thus, if these modifications and variations of the embodiments of the present disclosure fall within the scope of the claims of the present disclosure and equivalent technologies, the present disclosure also intends to include these modifications and variations.

What is claimed is:

1. An ultrasonic probe, comprising:
a plurality of transmitting transducers;
a plurality of receiving transducers, wherein each of the plurality of receiving transducers comprises a receiving component and an ultrasonic control circuit electrically connected to the receiving component, and a plurality of receiving components in the plurality of receiving transducers are distributed in an array; and
a plurality of scanning signal lines and a plurality of readout signal lines, wherein the scanning signal line is located in a row gap between adjacent receiving components, the readout signal line is located in a column gap between adjacent receiving components, receiving components in a same row are electrically connected with a same scanning signal line by means of corresponding ultrasonic control circuits, and receiving components in a same column are electrically connected with a same readout signal line by means of the corresponding ultrasonic control circuits.

2. The ultrasonic probe according to claim 1, wherein each of the plurality of transmitting transducers comprises: a first transmitting element which transmits a first acoustic wave signal, and at least one second transmitting element which transmits a second acoustic wave signal; wherein a frequency of the first acoustic wave signal is less than a frequency of the second acoustic wave signal;
wherein the receiving component comprises: a first receiving element which receives a third acoustic wave signal fed back according to the first acoustic wave signal, and a second receiving element which receives a fourth acoustic wave signal fed back according to the second acoustic wave signal.

3. The ultrasonic probe according to claim 2, wherein the first transmitting element and the second transmitting element are integrated in a same transmitting transducer; and
the first receiving element and the second receiving element are integrated in a same receiving transducer.

4. The ultrasonic probe according to claim 3, wherein each of the plurality of transmitting transducers comprises one first transmitting element, and a plurality of second transmitting elements; and
the plurality of the second transmitting elements are distributed around the one first transmitting element.

5. The ultrasonic probe according to claim 3, wherein the receiving component comprises:
a first substrate;
a first electrode on a side of the first substrate;
a piezoelectric film layer on a side of the first electrode facing away from the first substrate; and
a second electrode on a side of the piezoelectric film layer facing away from the first electrode.

6. The ultrasonic probe according to claim 5, wherein the ultrasonic control circuit is arranged between the first substrate and the first electrode; and
the ultrasonic control circuit comprises a first thin film transistor electrically connected with the receiving component, wherein the first electrode is electrically connected with a source of the first thin film transistor or a drain of the first thin film transistor.

7. The ultrasonic probe according to claim 5, wherein the first electrodes of different receiving components are independent of each other; and the second electrodes of the receiving components are an integrated structure.

8. The ultrasonic probe according to claim 3, wherein the plurality of transmitting transducers are distributed in an array; and
a distribution density of the plurality of transmitting transducers is smaller than a distribution density of the plurality of receiving components.

9. The ultrasonic probe according to claim 2, wherein the first transmitting element and the at least one second transmitting element are independent of each other; and the first receiving element and the second receiving element are independent of each other;
wherein the first transmitting element and the first receiving element are an integrated structure, and the at least one second transmitting element and the second receiving element are an integrated structure.

10. The ultrasonic probe according to claim 9, wherein the receiving component comprises:
a second substrate;
a third electrode on a side of the second substrate;
a cavity on a side of the third electrode facing away from the second substrate;
a diaphragm on a side of the cavity facing away from the third electrode; and
a fourth electrode on a side of the diaphragm facing away from the cavity;
wherein a dimension of the cavity of the first receiving element in a direction parallel to the second substrate is greater than a dimension of the cavity of the second receiving element in the direction parallel to the second substrate.

11. The ultrasonic probe of claim 10, wherein the ultrasonic control circuit is arranged between the second substrate and the third electrode; and
the ultrasonic control circuit comprises a second thin film transistor electrically connected with the receiving component, wherein the third electrode is electrically connected with a source of the second thin film transistor or a drain of the second thin film transistor.

12. The ultrasonic probe according to claim 10, wherein the third electrodes of different receiving components are independent of each other; and the fourth electrodes of the receiving components are an integrated structure.

13. The ultrasonic probe according to claim 9, wherein a distribution density of the plurality of second receiving elements is greater than a distribution density of the plurality of first receiving elements.

14. An ultrasonic apparatus, comprising the ultrasonic probe according to claim 1, wherein the ultrasonic apparatus further comprises a processor;
the processor is electrically connected with the transmitting transducers and the receiving transducers, and the processor is configured to provide excitation signals to the transmitting transducers, and receive feedback signals fed back by the receiving transducers.

15. A detection method for the ultrasonic probe according to claim 1, comprising:
controlling the transmitting transducers to transmit ultrasonic signals;
loading scan signals line by line onto the scanning signal lines; and
obtaining feedback signals received by the receiving components and fed back according to the ultrasonic signals by means of the readout signal lines.

16. The detection method according to claim 15, wherein operations of controlling the transmitting transducers to transmit ultrasonic signals; loading scan signals line by line onto the scanning signal lines; and obtaining feedback signals received by the receiving components and fed back according to the ultrasonic signals by means of the readout signal lines, comprise:
controlling a first transmitting element to transmit a first acoustic wave signal, loading a first scanning signal line by line to the scanning signal lines, and obtaining a third acoustic wave signal received by the first receiving element and fed back according to the first acoustic wave signal by means of the readout signal lines; wherein, the third acoustic wave signal comprises location information of a target object; and
controlling a second transmitting element to transmit a second acoustic wave signal to the target object, loading a second scanning signal line by line to the scanning signal lines, and obtaining a fourth acoustic wave signal received by the second receiving element and fed back according to the second acoustic wave signal by means of the readout signal lines, to perform imaging according to information of the received fourth acoustic wave signal.

17. The detection method according to claim 16, wherein the obtaining the fourth acoustic wave signal received by the second receiving element and fed back according to the second acoustic wave signal by means of the readout signal lines, comprises:
collecting the fourth acoustic wave signal obtained by the second receiving element every first time interval, wherein the first time interval is less than half of a period of the fourth acoustic wave signal; and
obtaining a plurality of fourth acoustic wave signals obtained by each second receiving element at respective first time intervals by means of the readout signal lines, to determine related information of the target object.

18. The detection method according to claim 17, wherein the determining the related information of the target object, comprises: obtaining coordinates of the target object according to a following formula:
$${t}_{n} \times v = \sqrt{{( X - xt )}^{2} + {( Y - yt )}^{2} + {( Z - zt )}^{2}} + \sqrt{{( X - xn )}^{2} + {( Y - yn )}^{2} + {( Z - zn )}^{2}} ;$$
wherein, Tₓ(xₜ, yₜ, zₜ) is a center position of the transmitting transducer, tₙ is a sampling moment, and (xₙ, yₙ, zₙ) is coordinates of the receiving transducer that receives the signal at a moment tₙ.

19. The ultrasonic apparatus according to claim 14, wherein each of the plurality of transmitting transducers comprises: a first transmitting element which transmits a first acoustic wave signal, and at least one second transmitting element which transmits a second acoustic wave signal; wherein a frequency of the first acoustic wave signal is less than a frequency of the second acoustic wave signal;
wherein the receiving component comprises: a first receiving element which receives a third acoustic wave signal fed back according to the first acoustic wave signal, and a second receiving element which receives a fourth acoustic wave signal fed back according to the second acoustic wave signal.

20. The ultrasonic apparatus according to claim 19, wherein the first transmitting element and the second transmitting element are integrated in a same transmitting transducer; and
the first receiving element and the second receiving element are integrated in a same receiving transducer.
